(19) United States
(12) Patent Application Publication
(10) Pub. No.: US 2024/0172999 A1
(43) Pub. Date: May 30, 2024

(54) DETERMINING DECISION SUPPORT OUTPUTS USING USER-SPECIFIC ANALYTE LEVEL CRITERIA

(71) Applicant: Dexcom, Inc., San Diego, CA (US)

(72) Inventors: Giada ACCIAROLI, San Diego, CA (US); Christopher R. HANNEMANN, San Diego, CA (US); Mark DERDZINSKI, Aliquippa, PA (US); Margaret A. CRAWFORD, San Diego, CA (US); Joost Herman VAN DER LINDEN, Afton, MN (US)

(21) Appl. No.: 18/498,548

(22) Filed: Oct. 31, 2023

Related U.S. Application Data
(60) Provisional application No. 63/385,581, filed on Nov. 30, 2022.

(51) Int. Cl. A61B 5/00; A61B 5/145

(52) U.S. Cl. CPC A61B 5/7275; A61B 5/14532; A61B 5/7267

(57) ABSTRACT

Systems, devices, and methods for determining decision support outputs using user-specific analyte level criteria for improving patients' health outcomes are provided. In one embodiment, a non-transitory computer readable storage medium storing a program is provided, the program comprising instructions that, when executed by at least one processor of a computing device, cause the at least one processor to perform operations including receiving sensor data generated by an analyte sensor configured to monitor at least one analyte, determining at least one analyte level criteria for the user for the at least one analyte; determining, using a decision support model, at least one decision support output based on the at least one analyte level criteria, and providing the at least one decision support output to the user.

### CROSS-REFERENCE TO RELATED APPLICATIONS

This application claims benefit of and priority to U.S. Provisional Application No. 63/385,581, filed Nov. 30, 2022, which is assigned to the assignee hereof and hereby expressly incorporated herein in its entirety as if fully set forth below and for all applicable purposes.

### BACKGROUND

### Field of Disclosure

This application generally relates to medical devices (e.g., analyte sensors), and more specifically to systems, devices, and methods for determining decision support outputs for improving patients' health outcomes.

### Description of the Related Technology

Diabetes is a metabolic condition relating to the production or use of insulin by the body. Insulin is a hormone that allows the body to use glucose for energy, or store glucose as fat.

When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), and kidney damage. Hyperglycemia can also lead to acute problems, such as diabetic ketoacidosis—a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

A diabetes patient can receive insulin to manage blood glucose levels. Insulin can be received, for example, through a manual injection with a needle. Wearable insulin pumps may also be utilized to receive insulin. Diet and exercise also affect blood glucose levels.

Diabetes conditions may be referred to as “Type 1” and “Type 2.” A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

This background is provided to introduce a brief context for the summary and detailed description that follow. This background is not intended to be an aid in determining the scope of the claimed subject matter nor be viewed as limiting the claimed subject matter to implementations that solve any or all of the disadvantages or problems presented above.

### BRIEF SUMMARY

The various embodiments of the present systems, devices, and methods for determining decision support outputs using user-specific analyte level criteria for improving patients' health outcomes comprise several features, no single one of which is solely responsible for their desirable attributes. Without limiting the scope of the present embodiments, their more prominent features will now be discussed below. After considering this discussion, and particularly after reading the section entitled “Detailed Description,” one will understand how the features of the present embodiments provide the advantages described here.

In a first aspect, a non-transitory computer readable storage medium storing a program is provided, the program comprising instructions that, when executed by at least one processor of a computing device, cause the at least one processor to perform operations including: receiving sensor data generated by an analyte sensor configured to monitor at least one analyte; determining at least one analyte level criteria for the user for the at least one analyte; determining, using a decision support model, at least one decision support output based on the at least one analyte level criteria; and providing the at least one decision support output to the user.

In an embodiment of the first aspect, the at least one analyte level criteria is an optimal level range for the at least one analyte.

In another embodiment of the first aspect, the optimal level range comprises a high-level analyte threshold that defines an upper boundary for the at least one analyte and a low-level analyte threshold that defines a lower boundary for the at least one analyte.

In another embodiment of the first aspect, the operations further comprise: receiving user input that indicates the high-level analyte threshold and the low-level analyte threshold, wherein the high-level analyte threshold and the low-level analyte threshold are determined based on the user input.

In another embodiment of the first aspect, the optimal level range is determined by: defining a threshold time period; and upon determining that the sensor data covers the threshold time period, generating trends using the sensor data to determine the high-level analyte threshold and the low-level analyte threshold.

In another embodiment of the first aspect, the optimal level range is determined by: defining a threshold time period; and upon determining that the sensor data does not cover the threshold time period, generating trends using sensor data of a cohort to determine the high-level analyte threshold and the low-level analyte threshold.

In another embodiment of the first aspect, the optimal level range is determined using a contextual multi-armed bandit algorithm.

In another embodiment of the first aspect, the at least one analyte level criteria is a risk tolerance profile for the user.

In another embodiment of the first aspect, the risk tolerance profile comprises a high-level risk tolerance threshold defining a user's willingness to risk having analyte levels exceed a recommended high analyte level range, and a low-level risk tolerance threshold defining the user's willingness to risk having the analyte levels fall below a recommended low analyte level range.

In another embodiment of the first aspect, the non-transitory computer readable storage medium further comprises instructions that, when executed by the at least one processor, further cause the at least one processor to: receive user input that indicates a user's risk tolerance levels; and determine the high-level risk threshold and the low-level risk threshold based on the user input.

In another embodiment of the first aspect, the user input includes the user's risk tolerance levels to a plurality of ranges for the recommended high analyte level range and a plurality of ranges for the recommended low analyte level range.

In another embodiment of the first aspect, the user input includes an acceptable number of high-level analyte events and low-level analyte events.

In another embodiment of the first aspect, the user input includes an acceptable time range for high-level analyte events and low-level analyte events.

In another embodiment of the first aspect, the non-transitory computer readable storage medium further comprises instructions that, when executed by the at least one processor, further cause the at least one processor to determine the risk tolerance profile based on a contextual multi-armed bandit algorithm.

In another embodiment of the first aspect, the non-transitory computer readable storage medium further comprises instructions that, when executed by the at least one processor, further cause the at least one processor to determine, using the decision support model, the at least one decision support output based on the at least one analyte level criteria by: determining a hyperparameter based on the optimal level range; and executing the decision support model using the hyperparameter.

In another embodiment of the first aspect, the non-transitory computer readable storage medium further comprises instructions that, when executed by the at least one processor, further cause the at least one processor to determine, using the decision support model, the at least one decision support output based on the at least one analyte level criteria by: determining a hyperparameter based on the risk tolerance profile; and executing the decision support model using the hyperparameter.

In another embodiment of the first aspect, the decision support model comprises a scoring sub-model and a decision sub-model.

In another embodiment of the first aspect, the non-transitory computer readable storage medium further comprises instructions that, when executed by the at least one processor, further cause the at least one processor to determine, using the decision support model, the at least one decision support output based on the at least one analyte level criteria by configuring the scoring sub-model to process the at least one analyte level criteria to determine at least one risk score for the user.

In another embodiment of the first aspect, the at least one risk score includes a predicted likelihood that the user experiences a particular condition in a future time period.

In another embodiment of the first aspect, the non-transitory computer readable storage medium further comprises instructions that, when executed by the at least one processor, further cause the at least one processor to determine the at least one decision support output using the decision support model by configuring the decision sub-model to select a decision support output for the user from a set of potential decision support outputs based on the at least one risk score.

In a second aspect, a method for determining decision support outputs using user-specific analyte level criteria is provided, the method comprising: receiving, by a data analysis module (DAM), sensor data generated by an analyte sensor configured to monitor at least one analyte; determining, by the DAM, at least one analyte level criteria for the user for the at least one analyte; determining, by the DAM, using a decision support model, at least one decision support output based on the at least one analyte level criteria; and providing, by the DAM, the at least one decision support output to the user.

In an embodiment of the second aspect, the at least one analyte level criteria is an optimal level range for the at least one analyte.

In another embodiment of the second aspect, the optimal level range comprises a high-level analyte threshold that defines an upper boundary for the at least one analyte and a low-level analyte threshold that defines a lower boundary for the at least one analyte.

In another embodiment of the second aspect, the method further comprises: receiving user input that indicates the high-level analyte threshold and the low-level analyte threshold, wherein the high-level analyte threshold and the low-level analyte threshold are determined based on the user input.

In another embodiment of the second aspect, the optimal level range is determined by: defining a threshold time period; and upon determining that the sensor data covers the threshold time period generating trends using the sensor data to determine the high-level analyte threshold and the low-level analyte threshold.

In another embodiment of the second aspect, the optimal level range is determined by: defining a threshold time period; and upon determining that the sensor data does not cover the threshold time period generating trends using sensor data of a cohort to determine the high-level analyte threshold and the low-level analyte threshold.

In another embodiment of the second aspect, the optimal level range is determined using a contextual multi-armed bandit algorithm.

In another embodiment of the second aspect, the at least one analyte level criteria is a risk tolerance profile for the user.

In another embodiment of the second aspect, the risk tolerance profile comprises a high-level risk tolerance threshold defining a user's willingness to risk having analyte levels exceed a recommended high analyte level range, and a low-level risk tolerance threshold defining the user's willingness to risk having the analyte levels fall below a recommended low analyte level range.

In another embodiment of the second aspect, the method further comprises: receiving user input that indicates a user's risk tolerance levels; and determining the high-level risk threshold and the low-level risk threshold based on the user input.

In another embodiment of the second aspect, the user input includes the user's risk tolerance levels to a plurality of ranges for the recommended high analyte level range and a plurality of ranges for the recommended low analyte level range.

In another embodiment of the second aspect, the user input includes an acceptable number of high-level analyte events and low-level analyte events.

In another embodiment of the second aspect, the user input includes an acceptable time range for high-level analyte events and low-level analyte events.

In another embodiment of the second aspect, the method further comprises determining the risk tolerance profile based on a contextual multi-armed bandit algorithm.

In another embodiment of the second aspect, the method further comprises determining, using the decision support model, the at least one decision support output based on the at least one analyte level criteria by: determining a hyperparameter based on the optimal level range; and executing the decision support model using the hyperparameter.

In another embodiment of the second aspect, the method further comprises determining, using the decision support model, the at least one decision support output based on the at least one analyte level criteria by: determining a hyperparameter based on the risk tolerance profile; and executing the decision support model using the hyperparameter.

In another embodiment of the second aspect, the decision support model comprises a scoring sub-model and a decision sub-model.

In another embodiment of the second aspect, the method further comprises determining, using the decision support model, the at least one decision support output based on the at least one analyte level criteria by configuring the scoring sub-model to process the at least one analyte level criteria to determine at least one risk score for the user.

In another embodiment of the second aspect, the at least one risk score includes a predicted likelihood that the user experiences a particular condition in a future time period.

In another embodiment of the second aspect, the method further comprises determining the at least one decision support output using the decision support model by configuring the decision sub-model to select a decision support output for the user from a set of potential decision support outputs based on the at least one risk score.

In a third aspect, computing device for determining decision support outputs using user-specific analyte level criteria is provided, the computing device comprising: a network interface; a processor operatively connected to the network interface; a memory storing a program comprising instructions that, when executed by the processor, cause the computing device to perform operations including: receive, using the network interface, sensor data generated by an analyte sensor configured to monitor at least one analyte; determine at least one analyte level criteria for the user for the at least one analyte; determine, using a decision support model, at least one decision support output based on the at least one analyte level criteria; and provide the at least one decision support output to the user.

In an embodiment of the third aspect, the at least one analyte level criteria is an optimal level range for the at least one analyte.

In another embodiment of the third aspect, the optimal level range comprises a high-level analyte threshold that defines an upper boundary for the at least one analyte and a low-level analyte threshold that defines a lower boundary for the at least one analyte.

In another embodiment of the third aspect, the operations further comprise: receiving user input that indicates the high-level analyte threshold and the low-level analyte threshold, wherein the high-level analyte threshold and the low-level analyte threshold based on the user input.

In another embodiment of the third aspect, the optimal level range is determined by: defining a threshold time period; and upon determining that the sensor data covers the threshold time period generating trends using the sensor data to determine the high-level analyte threshold and the low-level analyte threshold.

In another embodiment of the third aspect, the optimal level range is determined by: defining a threshold time period; and upon determining that the sensor data does not cover the threshold time period, then generating trends using sensor data of a cohort to determine the high-level analyte threshold and the low-level analyte threshold.

In another embodiment of the third aspect, the optimal level range is determined using a contextual multi-armed bandit algorithm.

In another embodiment of the third aspect, the at least one analyte level criteria is a risk tolerance profile for the user.

In another embodiment of the third aspect, the risk tolerance profile comprises a high-level risk tolerance threshold defining a user's willingness to risk having analyte levels exceed a recommended high analyte level range, and a low-level risk tolerance threshold defining the user's willingness to risk having the analyte levels fall below a recommended low analyte level range.

In another embodiment of the third aspect, the program comprises further instructions that, when executed by the processor, further cause the computing device to: receive user input that indicates a user's risk tolerance levels; and determine the high-level risk threshold and the low-level risk threshold based on the user input.

In another embodiment of the third aspect, the user input includes the user's risk tolerance levels to a plurality of ranges for the recommended high analyte level range and a plurality of ranges for the recommended low analyte level range.

In another embodiment of the third aspect, the user input includes an acceptable number of high-level analyte events and low-level analyte events.

In another embodiment of the third aspect, the user input includes an acceptable time range for high-level analyte events and low-level analyte events.

In another embodiment of the third aspect, the program comprises further instructions that, when executed by the processor, further cause the computing device to determine the risk tolerance profile based on a contextual multi-armed bandit algorithm.

In another embodiment of the third aspect, the program comprises further instructions that, when executed by the processor, further cause the computing device to determine, using the decision support model, the at least one decision support output based on the at least one analyte level criteria by: determining a hyperparameter based on the optimal level range; and executing the decision support model using the hyperparameter.

In another embodiment of the third aspect, the program comprises further instructions that, when executed by the processor, further cause the computing device to determine, using the decision support model, the at least one decision support output based on the at least one analyte level criteria by: determining a hyperparameter based on the risk tolerance profile; and executing the decision support model using the hyperparameter.

In another embodiment of the third aspect, the decision support model comprises a scoring sub-model and a decision sub-model.

In another embodiment of the third aspect, the program comprises further instructions that, when executed by the processor, further cause the computing device to determine, using the decision support model, the at least one decision support output based on the at least one analyte level criteria by configuring the scoring sub-model to process the at least one analyte level criteria to determine at least one risk score for the user.

In another embodiment of the third aspect, the at least one risk score includes a predicted likelihood that the user experiences a particular condition in a future time period.

In another embodiment of the third aspect, the program comprises further instructions that, when executed by the processor, further cause the computing device to determine the at least one decision support output using the decision support model by configuring the decision sub-model to select a decision support output for the user from a set of potential decision support outputs based on the at least one risk score.

### BRIEF DESCRIPTION OF THE DRAWINGS

FIG. 1A illustrates an example health monitoring and support system, in accordance with certain embodiments of the disclosure.

FIG. 1B illustrates a continuous analyte monitoring system in accordance with certain embodiments of the disclosure.

FIG. 2 illustrates example inputs and example metrics that are generated based on the inputs in accordance with certain embodiments of the disclosure.

FIG. 3 is a flow diagram illustrating a process for determining decision support outputs using user-specific analyte level criteria in accordance with certain embodiments of the disclosure.

FIG. 4 is a flow diagram illustrating a process for determining at least one analyte level criteria in accordance with certain embodiments of the disclosure.

FIG. 5 is a flow diagram illustrating a process for determining an optimal analyte level range for a user in accordance with certain embodiments of the disclosure.

FIG. 6 is a flow diagram illustrating a process for determining a risk tolerance profile for a user in accordance with certain embodiments of the disclosure.

FIG. 7 is a flow diagram illustrating a process for determining at least one decision support output using decision support models based on the at least one analyte level criteria in accordance with certain embodiments of the disclosure.

FIG. 8 is a flow diagram illustrating an exemplary operational flow of a contextual multi-armed bandit (CMAB) algorithm in accordance with certain embodiments of the disclosure.

FIG. 9 is a block diagram depicting a computing device configured to perform various processes for determining decision support outputs using user-specific analyte level criteria in accordance with certain embodiments of the disclosure.

### DETAILED DESCRIPTION

Portable and/or wearable health monitoring devices (also referred to herein as “health monitoring devices”) and mobile health applications (also referred to herein as “applications”), have rapidly become renowned for their capabilities to support user-centered care. For example, management of diabetes can present complex challenges for patients, clinicians, and caregivers, as a confluence of many factors can impact a patient's glucose level and glucose trends. To assist patients with better managing this condition, health monitoring devices (e.g., sensors and other types of monitoring and diagnostic devices) as well as a variety of mobile health applications (e.g., diabetes intervention software applications) have been developed. The wide dissemination of health monitoring devices and the increase in the development and distribution of mobile health applications has improved health management, and more specifically chronic disease management, in the healthcare domain. In particular, the use of mobile health applications in conjunction with these health monitoring devices, represents a more scalable and potentially more cost effective alternative to traditional interventions, offering a means of improving health and chronic disease management by expanding the reach of healthcare services and improving users' access to health-related information and interventions.

Mobile health applications enable users to be much more involved in the users' own medical care by granting them access to and control over their health information, which results in better patient satisfaction, and improvement in care and clinical outcomes. In particular, mobile health applications enable users to access, monitor, record, and update their health information regardless of physical constraints, such as time and location. Commercially available mobile health applications provide functions such as delivering health information, medication reminders, remote monitoring, and mobile analytics to increase a users' health literacy, encourage users to play a more active role in managing their health or disease, promote adherence to treatment, and other types of decision support guidance.

In particular, a variety of intervention applications have been developed to deliver guidance that may assist patients, caregivers, healthcare providers, or other users in improving lifestyle or clinical/patient outcomes by meeting a variety of challenges, such as analyte control, exercise, and/or other health factors. The term “analyte” as used herein refers without limitation to a substance or chemical constituent in the body or a biological sample. For example, diabetes intervention applications may assist patients, caregivers, healthcare providers, or other users in overnight glucose control (e.g., reduce incidence of hypoglycemic events or hyperglycemic excursions), glucose control during and after meals (e.g., use historical information and trends to increase glycemic control), hyperglycemia corrections (e.g., increase time in target zone while avoiding hypoglycemic events from over-correction), and/or hypoglycemia treatments (e.g., address hypoglycemia while avoiding “rebound” hyperglycemia), to name a few.

Mobile health applications may provide such assistance in some form of guidance to the user. For example, guidance may include a graphical summary of a user's data over time or a mobile notification to the user, where the notification is offered to inform, warn, and/or recommend some action to the user. As an example, the application may help a user respond to a health condition in real time by predicting events or trends and provide treatment recommendations to address occurring or potential events or trends in real time. This type of calculated guidance and support may relieve the cognitive burden on the user. Some mobile health applications may also allow data to be exported in various formats to be shared with third parties and/or to connect users directly with healthcare professionals for feedback, which may support an improved patient-professional dialogue. Thus, if utilized, mobile health applications may increase quality of care while at the same time offering the potential to reduce costs for healthcare systems.

Health monitoring devices enable real-time sensing and analysis of human physiological information for medical monitoring of various diseases and conditions, noninvasive medical care and administration of various therapies and medicine, and mobile health and wellness monitoring. Portability is a central feature of these health monitoring devices. Thus, where continuously utilized, these devices may provide many benefits including improved information access, reduced medical errors, improved quality of care, etc.

To be an effective support tool, it may be desirable for the health monitoring device and/or application to continuously, or frequently, capture the attention of users and to stimulate the users' interest to actively engage with the device and/or application. Engagement indicates the degree of interaction a user has with the technology, e.g., device and/or application. Because health technologies, including health monitoring devices and mobile health applications, are voluntary use systems, the extent of user engagement with these technologies is generally determined by the users' perceived quality of experience, ongoing benefit of usage, and/or consideration of viable alternatives to using the technology.

As discussed herein, unfortunately, health monitoring systems, including health monitoring devices and/or mobile health applications, designed to support the management of chronic diseases or health conditions have been plagued with low user engagement and high user attrition rates. A reason for low user engagement and/or high user attrition rates may include failure of health monitoring systems to provide individualized or personalized decision support outputs (e.g., information, recommendations, warnings, etc.). When a mobile health application fails to provide individualized and/or personalized decision support outputs, users of the application may find the outputs to be ineffective in enabling them to take a holistic approach to managing their health (e.g., diseases, conditions, fitness, etc.). Further, decision support outputs that are not tailored to an individual (i.e., not “individualized”) may result in sub-optimal health outcomes. In addition, decision support outputs that are not tailored to a cohort that the individual is a member (i.e., not “personalized”) may also result in sub-optimal health outcomes. Thus, user engagement associated with such mobile health applications may decrease and, thereby, user attrition rates may increase.

One reason many health management applications fail to provide individualized or personalized decision support outputs is that such applications do not effectively utilize user-specific preferences or data about optimal analyte levels and/or analyte risk thresholds. For example, a user may have personal preferences about what is an optimal glucose concentration range for the user, the user's willingness to risk going into hyperglycemia, and the user's willingness to risk going into hypoglycemia. As another example, a user may have personal preferences about what is an optimal potassium concentration range, the user's willingness to risk going into hyperpotassemia, and the user's willingness to risk going into hypopotassemia. To provide individualized and/or personalized decision support outputs, a mobile health application should determine decision support outputs in a manner that effectively utilizes user-specific preferences about optimal analyte levels and/or analyte risk tolerance thresholds. Otherwise, the decision support outputs may be less effective in providing individualized or personalized guidance to users, resulting in increased user attrition from the mobile health application.

As described above, a user (also referred to herein as a “patient”) may utilize one or more health monitoring devices, such as, but not limited to, one or more analyte sensors that are configured to measure and report analyte sensor outputs describing monitored analyte levels of the user. The user may then use a mobile health application configured to execute on a computing device (e.g., the user's display device such as, but not limited to, a smart phone, etc.) for receiving the sensor outputs and providing decision support outputs to the user based on the sensor outputs and/or user-specific analyte level criteria. For example, a decision support output may recommend one or more actions for the user to perform (e.g., a recommended sleeping pattern for the user), one or more warnings to the user about a predicted future physiological condition of the user (e.g., a warning that the user is likely to experience hyperglycemia in the next eight hours), etc. In some embodiments, the sensor outputs may be transmitted to one or more computing devices, one or more servers, and/or one or more user databases. In some embodiments, the one or more user databases may be separate from the one or more servers. In some embodiments, the one or more user databases may be an integral part of the one or more servers.

In certain embodiments, a computing device may utilize one or more decision support models in providing decision support outputs to the user. A decision support model may be a user-facing algorithm that determines a decision support output for a user based on various parameters such as, but not limited to, the sensor outputs and/or user-specific analyte level criteria. One example of a decision support model is a sleep advisor model that is configured to select a recommended sleep pattern for a user from a set of potential sleep patterns. Another example of a decision support model is a hyperglycemia warning model that is configured to warn a user if the hyperglycemia warning model determines that the user is at the risk of hyperglycemia in a future time period (e.g., in the next eight hours). An additional example of a decision support model is a gradual transition model (also referred to herein as a “nudging” model) that is configured to provide a sequence of recommended analyte levels for a user over a sequence of future time periods to help and encourage the user to gradually transition the user's analyte values from a current analyte value range to an optimal analyte value range.

Certain embodiments described herein enable determining a decision support output using a decision support model whose operations are determined based on a set of analyte level criteria for the user. An analyte level criterion for a user may be one or more values that describe an aspect of an acceptable analyte level range (e.g., an acceptable glucose concentration range) for the user, where the value(s) may be selected for the user specifically (i.e., “individualized”) or for a cohort that includes the user (i.e., “personalized”). Examples of analyte level criteria include high-level analyte thresholds (e.g., hyperglycemia thresholds), low-level analyte thresholds (e.g., hypoglycemia thresholds), high-level risk tolerance thresholds (e.g., hyperglycemia risk tolerance thresholds), and low-level risk tolerance thresholds (e.g., hypoglycemia risk tolerance thresholds).

A high-level analyte threshold may define the upper boundary of a recommended analyte level range for a user. A low-level analyte threshold may define the lower boundary of the recommended analyte level range for the user. A high-level risk tolerance threshold may define the extent of the user's willingness to risk having analyte levels that exceed the recommended analyte level range for the user. A low-level risk tolerance threshold may define the extent of the user's willingness to risk having analyte levels that fall below the recommended analyte level range for the user. In some embodiments, a user may have more than one high-level risk tolerance threshold and/or low-level risk tolerance threshold. For example, the user may have a first high-risk tolerance threshold for a first analyte level range that exceeds the recommended analyte level range for the user and a second high-risk tolerance threshold for a second analyte level range that exceeds the recommended analyte level range for the user. As another example, the user may have a first low-risk tolerance threshold for a first analyte level range that falls below the recommended analyte level range for the user and a second low-risk tolerance threshold for a second analyte level range that falls below the recommended analyte level range for the user.

To determine decision support outputs for a user, a computing device may be configured to first determine one or more analyte level criteria for the user. In some embodiments, the mobile health management application may use one or more decision support models and the determined one or more analyte level criteria to determine the decision support outputs. There may be various techniques for using analyte level criteria of a user in conjunction with a decision support model to determine a decision support output. For example, in a first technique, one or more analyte level criteria for a user may be used to determine user-specific hyperparameters for a decision support model. User-specific hyperparameters may be used by the decision support model to determine decision support outputs with respect to the specific user that is associated with the hyperparameters. Using this first technique, the decision support model may be able to use different user-specific hyperparameters to determine decision support outputs for different users, thereby increasing the adaptability and the predictive accuracy of the decision support model. An example of a user-specific hyperparameter that can be determined based on the analyte level criteria is a user-specific hyperglycemia threshold for a hyperglycemia warning model. A user-specific hyperglycemia threshold can be used by the hyperglycemia warning model to determine whether to warn the corresponding user about a likelihood of hyperglycemia occurring in the future. In this example, the hyperglycemia warning model is an example of a decision support model, the user-specific hyperglycemia threshold is an example of a user-specific hyperparameter that is determined based on the analyte level criteria for the corresponding user, and the warning determination is an example of a decision support output that is determined for the corresponding user.

A second technique for using analyte level criteria of a user in conjunction with a decision support model may involve providing the analyte level criteria as model inputs to the decision support model to determine a decision support output for the user. For example, analyte level criteria for a user may be used to determine an optimal analyte level range for the user. Once the optimal analyte level range for the user is determined, the range may be provided as an input to a gradual transition model. The gradual transition model may use the optimal analyte range for the user to determine a sequence of recommended analyte levels for the user.

In both of the techniques described above, the analyte level criteria for a user may be integrated into the operational logic of a decision support model to determine a decision support output for the user. Because the embodiments described herein enable integrating user-specific analyte level criteria into the operational logic of the decision support model, the resulting decision support output is more likely to respond to the specific needs of the user and, thereby, be effective for the user. As described above, providing ineffective decision support outputs to users leads to higher user attrition rates from mobile health applications. By enabling mobile health applications to provide more effective decision support outputs, certain embodiments described herein are likely to increase user engagement with those applications and, thereby, decrease user attrition from those applications.

Further, although the description and examples below are drawn to a glucose monitoring sensor capable of measuring the concentration of glucose in a host, the systems, devices, and methods of the embodiments described herein can be used in conjunction with any type of analyte sensor for any measurable analyte. The system, devices, and methods of the embodiment described herein may be used in conjunction with any health-related application that is provided to the user to improve the user's health. For example, a health-related application may help the user with treating a certain disease or just help with improving the health of a user who is not necessarily diagnosed with a disease.

### Example System with a Decision Support Engine for Determining Decision Support Outputs Using User-Specific Analyte Level Criteria

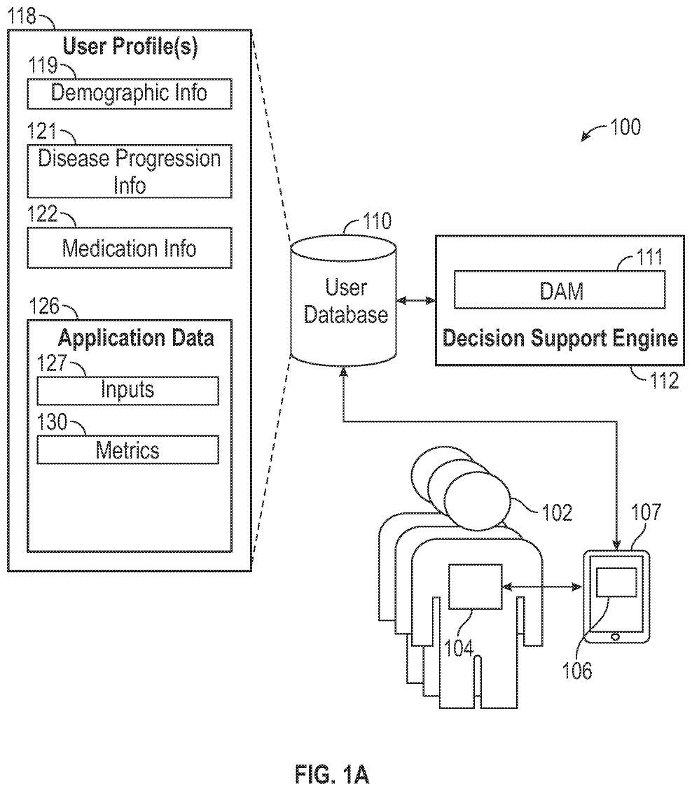

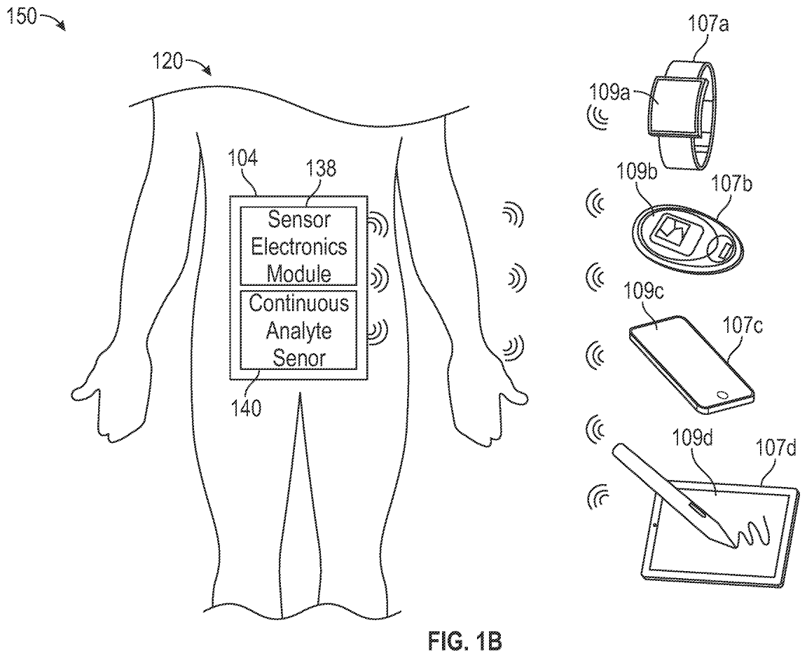

FIG. 1A illustrates an example health monitoring and support system in accordance with certain embodiments of the disclosure. The health monitoring and support system 100 may be utilized for monitoring user health, determining decision support outputs using user-specific analyte level criteria, and providing decision support to users associated with system 100. Each user of system 100, such as user 102, may interact with a mobile health application, such as mobile health application (“application”) 106 (e.g., a diabetes intervention application that provides decision support guidance), and/or a health monitoring device, such as an analyte monitoring system 104. User 102, in certain embodiments, may be the patient or, in some cases, the patient's caregiver. In the embodiments described herein, the user is assumed to be the patient for simplicity only, but is not so limited. As shown, system 100 may include an analyte monitoring system 104, a mobile device 107 that executes application 106, a decision support engine 112 (including a DAM 111), and a user database 110.

Analyte monitoring system 104 may be configured to generate analyte measurements for the user 102, e.g., on a continuous basis, and transmit the analyte measurements to the mobile device 107 for use by application 106. In some embodiments, the analyte monitoring system 104 may transmit the analyte measurements to the mobile device 107 through a wireless connection (e.g., Bluetooth connection). In certain embodiments, mobile device 107 is a smart phone. However, in certain embodiments, mobile device 107 may instead be any other type of computing device such as a laptop computer, a smart watch, a tablet, or any other computing device capable of executing application 106.

Note that, while in certain examples the analyte monitoring system 104 is assumed to be a glucose monitoring system, analyte monitoring system 104 may operate to monitor one or more additional or alternative analytes. As discussed, the term “analyte” as used herein is a broad term, and is to be given its ordinary and customary meaning to a person of ordinary skill in the art (and is not to be limited to a special or customized meaning), and refers without limitation to a substance or chemical constituent in the body or a biological sample (e.g., bodily fluids, including, blood, serum, plasma, interstitial fluid, cerebral spinal fluid, lymph fluid, ocular fluid, saliva, oral fluid, urine, excretions, or exudates). Analytes can include naturally occurring substances, artificial substances, metabolites, and/or reaction products. In some embodiments, the analyte for measurement by the sensing regions, devices, and methods is albumin, alkaline phosphatase, alanine transaminase, aspartate aminotransferase, bilirubin, blood urea nitrogen, calcium, CO2, chloride, creatinine, glucose, gamma-glutamyl transpeptidase, hematocrit, lactate, lactate dehydrogenase, magnesium, oxygen, pH, phosphorus, potassium, sodium, total protein, uric acid, metabolic markers, and drugs.

Other analytes are contemplated as well, including but not limited to acetaminophen, dopamine, ephedrine, terbutaline, ascorbate, uric acid, oxygen, d-amino acid oxidase, plasma amine oxidase, xanthine oxidase, NADPH oxidase, alcohol oxidase, alcohol dehydrogenase, pyruvate dehydrogenase, diols, Ros, NO, bilirubin, cholesterol, triglycerides, gentisic acid, ibuprophen, L-Dopa, methyl dopa, salicylates, tetracycline, tolazamide, tolbutamide, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F. D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free ß-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprac, Mycoplasma pneumoniac, Myoglobin, Onchocerca volvulus, parainfluenza virus, Plasmodium falciparum, poliovirus, Pseudomonas acruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments.

The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), histamine, Advanced Glycation End Products (AGEs) and 5-hydroxyindoleacetic acid (FHIAA).

Application 106 may be a mobile health application that is configured to receive and analyze analyte measurements from the analyte monitoring system 104. In some embodiments, application 106 may transmit analyte measurements received from the analyte monitoring system 104 to a user database 110 (and/or the decision support engine 112), and the user database (and/or the decision support engine 112) may store the analyte measurements in a user profile 118 of user 102 for processing and analysis as well as for use by the decision support engine 112 to determine decision support outputs using user-specific analyte level criteria and provide decision support outputs such as, but not limited to, recommendations and/or guidance to the user 102 via the application 106. In some embodiments, application 106 may store the analyte measurements in a user profile 118 of user 102 locally for processing and analysis as well as for use by the decision support engine 112 to determine decision support outputs using user-specific analyte level criteria and provide decision support outputs such as, but not limited to, recommendations and/or guidance to the user 102.

In certain embodiments, decision support engine 112 refers to a set of software instructions with one or more software modules, including a data analysis module (DAM) 111. In some embodiments, decision support engine 112 executes entirely on one or more computing devices in a private or a public cloud. In some other embodiments, decision support engine 112 executes partially on one or more local devices, such as mobile device 107, and partially on one or more computing devices in a private or a public cloud. In some other embodiments, decision support engine 112 executes entirely on one or more local devices, such as mobile device 107.

As discussed in more detail herein, decision support engine 112, may determine decision support outputs using user-specific analyte level criteria and provide decision support outputs (e.g., decision support recommendations, etc.) to the user 102 via the application 106. For example, the decision support engine 112 may determine an individualized or personalized decision support output for a user using user-specific analyte level criteria by determining a set of analyte level criteria for the user 102 and executing a decision support model based on the set of analyte level criteria to determine the decision support output. In certain embodiments, to determine an individualized decision support output for the user 102, the set of analyte level criteria may be specifically determined for the user 102. In some embodiments, to determine a personalized decision support output for the user, the set of analyte level criteria may be determined for a cohort that includes the user 102. As used herein, a “cohort” may be a set of “similar” users, with similar demographics, health history and/or other characteristics that influence outcomes. Further, the decision support engine 112 may execute a decision support model based on the set of analyte level criteria to determine the decision support output. To execute the decision support model based on the set of analyte level criteria, the analyte level criteria may be used to determine at least one of input data for the decision support model or user-specific hyperparameters of the decision support model, as further described below.

In some embodiments, the decision support engine 112 may determine decision support outputs using user-specific analyte level criteria based on information including, but not limited to, information included in the user profile 118 stored in the user database 110. In some embodiments, the user profile 118 may include information collected about the user from the application 106, as further described below.

In certain embodiments, DAM 111 of decision support engine 112 may be configured to receive and/or process a set of inputs 127 (described in more detail below) (also referred to herein as “input data”) to determine one or more metrics 130 (also referred to herein as “metrics data”) that may then be used by decision support engine 112 in determining decision support outputs and providing the decision support outputs to the user 102. Inputs 127 may be stored in the user profile 118 in the user database 110. DAM 111 can fetch inputs 127 from the user database 110 and compute a plurality of metrics 130 which can then be stored as application data 126 in the user profile 118. Such metrics 130 may include health-related metrics.

In certain embodiments, application 106 is configured to take as input information relating to user 102 and store the information in a user profile 118 for user 102 in user database 110. For example, application 106 may obtain and record user 102's demographic info 119, disease progression info 121, and/or medication info 122 in user profile 118. In certain embodiments, demographic info 119 may include one or more of the user's age, body mass index (BMI), ethnicity, gender, etc. In certain embodiments, disease progression info 121 may include information about the user 102's disease, such as, for diabetes, whether the user is Type I, Type II, pre-diabetes, or whether the user has gestational diabetes. In certain embodiments, disease progression info 121 also includes the length of time since diagnosis, the level of disease control, level of compliance with disease management therapy, predicted pancreatic function, other types of diagnosis (e.g., heart disease, obesity) or measures of health (e.g., heart rate, exercise, stress, sleep, etc.), and/or the like. In certain embodiments, medication regimen info 122 may include information about the amount and type of medication taken by user 102, such as insulin or non-insulin diabetes medications and/or non-diabetes medication taken by user 102.

In certain embodiments, application 106 may obtain demographic info 119, disease progression info 121, and/or medication info 122 from the user 102 in the form of user input or from other sources. In certain embodiments, as some of this information changes, application 106 may receive updates from the user 102 or from other sources. In certain embodiments, user profile 118 associated with the user 102, as well as other user profiles associated with other users are stored in a user database 110, which is accessible to application 106, as well as to the decision support engine 112, over one or more networks (not shown). In certain embodiments, application 106 collects inputs 127 through user 102 input and/or a plurality of other sources, including analyte monitoring system 104, other applications running on mobile device 107, and/or one or more other sensors and devices. In certain embodiments, such sensors and devices include one or more of, but are not limited to, an insulin pump, other types of analyte sensors, sensors or devices provided by mobile device 107 (e.g., accelerometer, camera, global positioning system (GPS), heart rate monitor, etc.) or other user accessories (e.g., a smart watch), or any other sensors or devices that provide relevant information about the user 102. In certain embodiments, user profile 118 also stores application configuration information indicating the current configuration of application 106, including its features and settings.

User database 110, in some embodiments, refers to a storage server that may operate in a public or private cloud. User database 110 may be implemented as any type of datastore, such as relational databases, non-relational databases, key-value datastores, file systems including hierarchical file systems, and the like. In some exemplary implementations, user database 110 is distributed. For example, user database 110 may comprise a plurality of persistent storage devices, which are distributed. Furthermore, user database 110 may be replicated so that the storage devices are geographically dispersed.

User database 110 may include other user profiles 118 associated with a plurality of other users served by health monitoring and decision support system 100. More particularly, similar to the operations performed with respect to the user 102, the operations performed with respect to these other users may utilize an analyte monitoring system, such as analyte monitoring system 104, and also interact with the same application 106, copies of which execute on the respective mobile devices of the other users 102. For such users, user profiles 118 are similarly created and stored in user database 110.

FIG. 1B illustrates a continuous analyte monitoring system in accordance with certain embodiments of the disclosure. The diagram 150 illustrates an example of an analyte monitoring system 104. In the example of FIG. 1B, the analyte monitoring system 104 is a glucose monitoring system. However, as described above, analyte monitoring system 104 may be configured for measuring any other analytes or a combination of multiple analytes. FIG. 1B illustrates a number of mobile devices 107a, 107b, 107c, and 107d (individually referred to as mobile device 107 and collectively referred to as mobile devices 107). Note that mobile device 107 of FIG. 1A may be any one of mobile devices 107a, 107b, 107c, or 107d. In other words, any one of mobile devices 107a, 107b, 107c, or 107d may be configured to execute application 106. The analyte monitoring system 104 may be communicatively coupled to mobile devices 107a, 107b, 107c, and/or 107d.

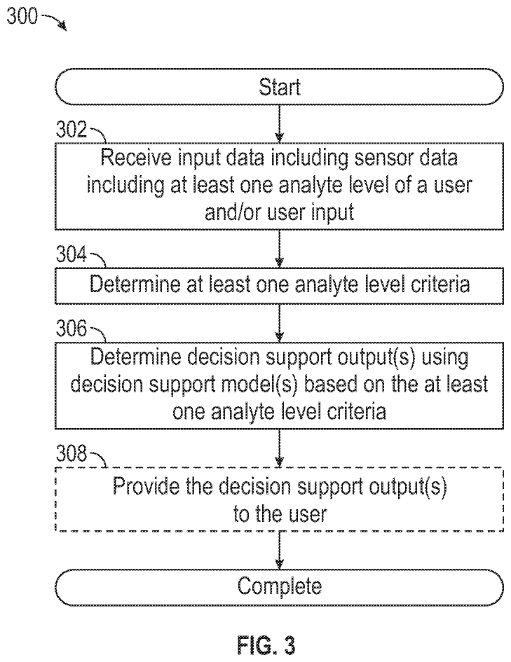

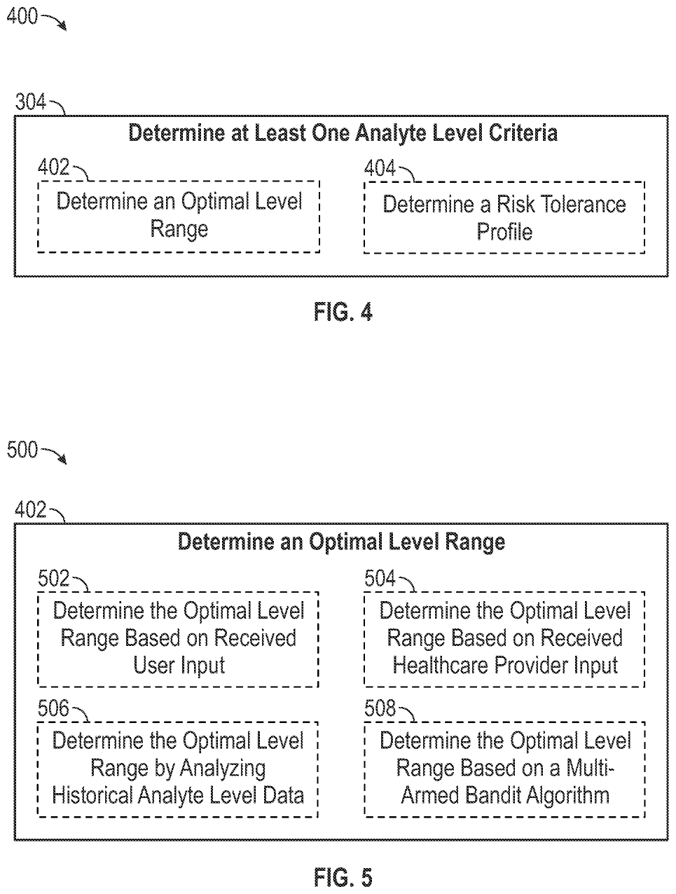

By way of an overview and an example, the analyte monitoring system 104 may be implemented as an encapsulated microcontroller that makes sensor measurements, generates analyte data (e.g., by calculating values for continuous glucose monitoring data), and engages in wireless communications (e.g., via Bluetooth and/or other wireless protocols) to send such data to remote devices, such as mobile devices 107. Paragraphs [0137]-[0140] and FIGS. 3A, 3B, and 4 of U.S. Patent Application Publication No. 2019/0336053 further describe an on-skin sensor assembly that, in certain embodiments, may be used in connection with analyte monitoring system 104. Paragraphs [0137]-[0140] and FIGS. 3A, 3B, and 4 of U.S. Patent Application Publication No. 2019/0336053 are incorporated herein by reference.

In certain embodiments, analyte monitoring system 104 includes an analyte sensor electronics module 138 and a continuous analyte sensor 140 (e.g., a glucose sensor) associated with the analyte sensor electronics module 138. In certain embodiments, analyte sensor electronics module 138 includes electronic circuitry associated with measuring and processing analyte sensor data (also referred to herein as “sensor outputs”) or information, including algorithms associated with processing and/or calibration of the analyte sensor data/information. Analyte sensor electronics module 138 may be physically/mechanically connected to the analyte sensor 140 and can be integral with (e.g., non-releasably attached to) or releasably attachable to the analyte sensor 140.

Analyte sensor electronics module 138 may also be electrically coupled to analyte sensor 140, such that the components may be electromechanically coupled to one another. Analyte sensor electronics module 138 may include hardware, firmware, and/or software that enable measurement and/or estimation of levels of the analyte in the user via analyte sensor 140 (e.g., which may be/include a glucose sensor). For example, analyte sensor electronics module 138 can include one or more potentiostats, a power source for providing power to analyte sensor 140, other components useful for signal processing and data storage, and a telemetry module for transmitting data from the sensor electronics module to various devices including, but not limited to, one or more display devices (e.g., the user's mobile device 107), user database 110, decision support engine 112, etc. Electronics can be affixed to a printed circuit board (PCB) within analyte monitoring system 104, or platform or the like, and can take a variety of forms. For example, the electronics can take the form of an integrated circuit (IC), such as an Application-Specific Integrated Circuit (ASIC), a microcontroller, a processor, and/or a state machine.

Analyte sensor electronics module 138 may include sensor electronics that are configured to process sensor information, such as sensor data, and generate transformed sensor data and displayable sensor information. Examples of systems and methods for processing sensor analyte data are described in more detail herein and in U.S. Pat. Nos. 7,310,544 and 6,931,327 and U.S. Patent Application Publication Nos. 2005/0043598, 2007/0032706, 2007/0016381, 2008/0033254, 2005/0203360, 2005/0154271, 2005/0192557, 2006/0222566, 2007/0203966 and 2007/0208245, all of which are incorporated herein by reference in their entireties.

Analyte sensor 140 is configured to measure a concentration or level of the analyte in the user 102. The term analyte is further defined by paragraph of U.S. App. No. 2019/0336053. Paragraph of U.S. App. No. 2019/0336053 is incorporated herein by reference. In some embodiments, analyte sensor 140 comprises a continuous analyte sensor, such as a subcutaneous, transdermal (e.g., transcutaneous), or intravascular device. In some embodiments, analyte sensor 140 can analyze a plurality of intermittent blood samples. Analyte sensor 140 can use any method of analyte-measurement, including enzymatic, chemical, physical, electrochemical, spectrophotometric, polarimetric, calorimetric, iontophoretic, radiometric, immunochemical, and the like. Additional details relating to a continuous analyte sensor, such as a continuous glucose sensor, are provided in paragraphs [0072]-[0076] of U.S. Pat. No. 9,445,445. Paragraphs [0072]-[0076] of U.S. Pat. No. 9,445,445 are incorporated herein by reference.

With further reference to FIG. 1B, mobile devices 107 can be configured for displaying (and/or alarming) displayable sensor information that may be transmitted by sensor electronics module 138 (e.g., in a customized data package that is transmitted to the display devices based on their respective preferences). Each of mobile devices 107a, 107b, 107c, and/or 107d may respectively include a display such as touchscreen display 109a, 109b, 109c, and/or 109d for displaying a graphical user interface (e.g., of application 106) for presenting sensor information and/or analyte data to user 102 and/or receiving inputs from user 102. In certain embodiments, the mobile devices 107 may include other types of user interfaces such as voice user interface instead of or in addition to a touchscreen display for communicating sensor information to user 102 of the mobile device 107 and/or receiving user inputs. In certain embodiments, one, some, or all of mobile devices 107 may be configured to display or otherwise communicate the sensor information as it is communicated from sensor electronics module 138 (e.g., in a data package that is transmitted to respective display devices), without any additional prospective processing required for calibration and/or real-time display of the sensor data.

The mobile devices 107 may include a custom or proprietary display device, for example, analyte display device 107b, especially designed for displaying certain types of displayable sensor information associated with analyte data received from sensor electronics module 138 (e.g., a numerical value and/or an arrow, in certain embodiments). In certain embodiments, one of the mobile devices 107 includes a mobile phone, such as a smartphone that uses an Android, IOS, or another operating system configured to display a graphical representation of the continuous sensor data (e.g., including current and/or historic data).

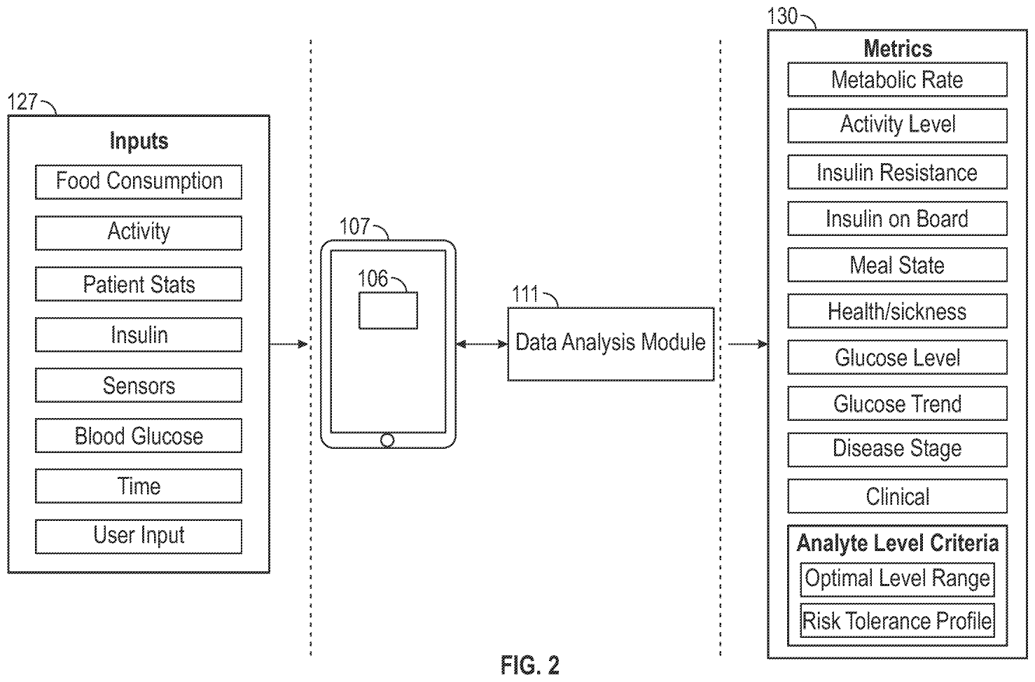

Example inputs and example metrics that are generated based on the inputs in accordance with certain embodiments of the disclosure are illustrated in FIG. 2. FIG. 2 illustrates example inputs 127 on the left, application 106 and DAM 111 in the middle, and example metrics 130 on the right. In certain embodiments, application 106 may obtain inputs 127 through one or more channels (e.g., manual user input, sensors, various applications executing on mobile device 107, etc.). Inputs 127 may be further processed by DAM 111 to output a plurality of metrics, such as metrics 130, which may similarly be used by features 1-N of application 106 to provide guidance to the user. In certain embodiments, inputs 127 and metrics 130 may also be used as contextual information by the DAM 111 and/or any computing device in the system 100 to perform various actions such as, but not limited to identifying a cohort and/or defining various sub-groups within a user cohort. Further, inputs (e.g., inputs 127) and metrics (e.g., metrics 130) may be used by the DAM 111 and/or any computing device in the system 100 to perform various processes in determining decision support outputs using user-specific analyte level criteria, as further described below. Any of inputs 127 may be used for computing any of metrics 130. In certain embodiments, each one of metrics 130 may correspond to one or more values, e.g., discrete numerical values, ranges, or qualitative values (high/medium/low or stable/unstable).

In certain embodiments, inputs 127 include food consumption information. Food consumption information may include information about one or more of meals, snacks, and/or beverages, such as one or more of the size, content (carbohydrate, fat, protein, etc.), sequence of consumption, and time of consumption. In certain embodiments, food consumption may be provided by the user through manual entry, by providing a photograph through an application that is configured to recognize food types and quantities, and/or by scanning a bar code or menu. In various examples, meal size may be manually entered as one or more of calories, quantity (e.g., ‘three cookies’), menu items (e.g., ‘Royale with Cheese’), and/or food exchanges (1 fruit, 1 dairy). In some examples, meals may also be entered with the user's typical items or combinations for this time or context (e.g., workday breakfast at home, weekend brunch at restaurant). In some examples, meal information may be received via a convenient user interface provided by application 106.

In certain embodiments, inputs 127 include activity information. Activity information may be provided, for example, by an accelerometer sensor on a wearable device such as a watch, fitness tracker, and/or patch. In certain embodiments, activity information may also be provided through manual input by user 102.

In certain embodiments, inputs 127 include patient statistics, such as one or more of age, height, weight, body mass index, body composition (e.g., % body fat), stature, build, or other information. Patient statistics may be provided through a user interface, by interfacing with an electronic source such as an electronic medical record, and/or from measurement devices. The measurement devices may include one or more of a wireless, e.g., Bluetooth-enabled, weight scale and/or camera, which may, for example, communicate with the mobile device 107 to provide patient data.

In certain embodiments, inputs 127 include information relating to the user's insulin delivery. Such information may be received, via a wireless connection on a smart pen, via user input, and/or from an insulin pump. Insulin delivery information may include one or more of insulin volume, time of delivery, etc. Other configurations, such as insulin action time or duration of insulin action, may also be received as inputs.

In certain embodiments, inputs 127 include information received from sensors, such as physiologic sensors, which may detect one or more of heart rate, respiration, oxygen saturation, body temperature, etc. (e.g., to detect illness).

In certain embodiments, inputs 127 include glucose information. Such information may be provided as input, for example through analyte monitoring system 104. In certain embodiments, blood glucose information may be received from one or more of smart pill dispensers that track when the user takes medicine, a blood ketone meter, a laboratory-measured, or estimated A1C, other measures of long-term control, or sensors that measure peripheral neuropathy using tactile response, such as by using haptic features of a smartphone, or a specialty device.

In certain embodiments, inputs 127 include time, such as time of day, or time from a real-time clock.

As described above, in certain embodiments, DAM 111 determines or computes metrics 130 based on inputs 127 associated with user 102. An example list of metrics 130 is illustrated in FIG. 2. In certain embodiments, metrics 130 determined or computed by DAM 111 include metabolic rate. Metabolic rate is a metric that may indicate or include a basal metabolic rate (e.g., energy consumed at rest) and/or an active metabolism, e.g., energy consumed by activity, such as exercise or exertion. In some examples, basal metabolic rate and active metabolism may be tracked as separate metric. In certain embodiments, the metabolic rate may be calculated by DAM 111 based on one or more of inputs 127, such as one or more of activity information, sensor input, time, user input, etc.

In certain embodiments, metrics 130 determined or computed by DAM 111 include an activity level metric. The activity level metric may indicate a level of activity of the user. In certain embodiments, the activity level metric be determined, for example based on input from an activity sensor or other physiologic sensors. In certain embodiments, the activity level metric may be calculated by DAM 111 based on one or more of inputs 210, such as one or more of activity information, sensor input, time, user input, etc.

In certain embodiments, metrics 130 determined or computed by DAM 111 include an insulin sensitive metric (also referred to herein as an “insulin resistance”). The insulin sensitivity metric may be determined using historical data, real-time data, or a combination thereof, and may, for example, be based upon one or more inputs 127, such as one or more of food consumption information, blood glucose information, insulin delivery information, the resulting glucose levels, etc. In certain embodiments, the insulin on board metric may be determined using insulin delivery information, and/or known or learned (e.g., from patient data) insulin time action profiles, which may account for both basal metabolic rate (e.g., update of insulin to maintain operation of the body) and insulin usage driven by activity or food consumption.

In certain embodiments, metrics 130 determined or computed by DAM 111 include a meal state metric. The meal state metric may indicate the state the user is in with respect to food consumption. For example, the meal state may indicate whether the user is in one of a fasting state, pre-meal state, eating state, post-meal response state, or stable state. In certain embodiments, the meal state may also indicate nourishment on board, e.g., meals, snacks, or beverages consumed, and may be determined, for example from food consumption information, time of meal information, and/or digestive rate information, which may be correlated to food type, quantity, and/or sequence (e.g., which food/beverage was eaten first.).

In certain embodiments, metrics 130 determined or computed by DAM 111 include health and sickness metrics. Health and sickness metrics may be determined, for example, based on one or more of user input (e.g., pregnancy information or known sickness information), from physiologic sensors (e.g., temperature), activity sensors, or a combination thereof. In certain embodiments, based on the values of the health and sickness metrics, for example, the user's state may be defined as being one or more of healthy, ill, rested, or exhausted.

In certain embodiments, metrics 130 determined or computed by DAM 111 include glucose level metrics. Glucose level metrics may be determined from sensor information (e.g., blood glucose information obtained from analyte monitoring system 104). In some examples, a glucose level metric may also be determined, for example, based upon historical information about glucose levels in particular situations, e.g., given a combination of food consumption, insulin, and/or activity. In certain embodiments, a blood glucose trend may be determined based on the glucose level over a certain period of time.

In certain embodiments, metrics 130 determined or computed by DAM 111 include a disease stage. For example disease stages for Type II diabetics may include a pre-diabetic stage, an oral treatment stage, and a basal insulin treatment stage. In certain embodiments, degree of glycemic control (not shown) may also be determined as an outcome metric, and may be based, for example, on one or more of glucose levels, variation in glucose level, or insulin dosing patterns.

In certain embodiments, metrics 130 determined or computed by DAM 111 include clinical metrics. Clinical metrics generally indicate a clinical state a user is in with respect to one or more conditions of the user, such as diabetes. For example, in the case of diabetes, clinical metrics may be determined based on glycemic measurements, including one or more of A1C. trends in A1C, time in range, time spent below a threshold level, time spent above a threshold level, and/or other metrics derived from blood glucose values. In certain embodiments, clinical metrics may also include one or more of estimated A1C, glycemic variability, hypoglycemia, and/or health indicator (time magnitude out of target zone).

In certain embodiments, metrics 130 determined or computed by DAM 111 may include at least one analyte level criteria, as further described below. In some embodiments, the at least one analyte level criteria may include an optimal level range of an analyte and a risk tolerance profile for a user. As further described below, an optimal level range and a risk tolerance profile for a user may be determined based on inputs 127 (e.g., received user input, received healthcare provider input). In some embodiments, an optimal level range and a risk tolerance profile for a user may be determined based on metrics 130 such as, but not limited, trends in historical analyte level data (e.g., glucose trends). In some embodiments, an optimal level range and a risk tolerance profile for a user may be determined based on various algorithms that may utilize inputs 127 and metrics 130, as further described below. An optimal level range and a risk tolerance profile are described in more detail below.

As discussed herein, DAM 111 and/or application 106 may be implemented in one or more computing devices to perform various processes for determining decision support outputs using user-specific analyte level criteria. For example, such processes may include (1) determining at least one analyte level criteria for a user and (2) determining decision support output(s) using decision support model(s) based on the at least one analyte level criteria, as further described below. In some embodiments, determining the at least one analyte level criteria for a user may include determining an optimal level range and/or determining a risk tolerance profile. In some embodiments, determining the decision support output(s) using the decision support model(s) based on the at least one analyte level criteria may include determining a hyperparameter and/or executing the decision support model(s) using the at least one analyte, as further described below.

### Example Processes for Determining Decision Support Outputs Using User-Specific Analyte Level Criteria

FIG. 3 is a flow diagram illustrating a process 300 for determining decision support outputs using user-specific analyte level criteria in accordance with certain embodiments of the disclosure. In certain embodiments, the process 300 may be performed by one or more computing devices, a system (e.g., health monitoring and support system 100), etc. For example, process 300 may be performed using an analyte monitoring system 104, application 106, user data 110, data analysis module 111, and/or other components of health monitoring and support system 100 illustrated in FIG. 1A. In some embodiments, the process 300 may include receiving (block 302) input data (e.g., input 127) such as, not limited to, sensor data generated by one or more analyte sensors (e.g., continuous analyte sensor 140) that are configured to measure one or more analyte levels of a user. In some embodiments, the input data may include user inputs and/or healthcare provider inputs. As further described below, the process 300 may include determining (block 304) at least one analyte level criteria and determining (block 306), using one or more decision support models, one or more decision support outputs based on the at least one analyte level criteria. In addition, the process 300 may include providing (308) the one or more decision support outputs to the user.

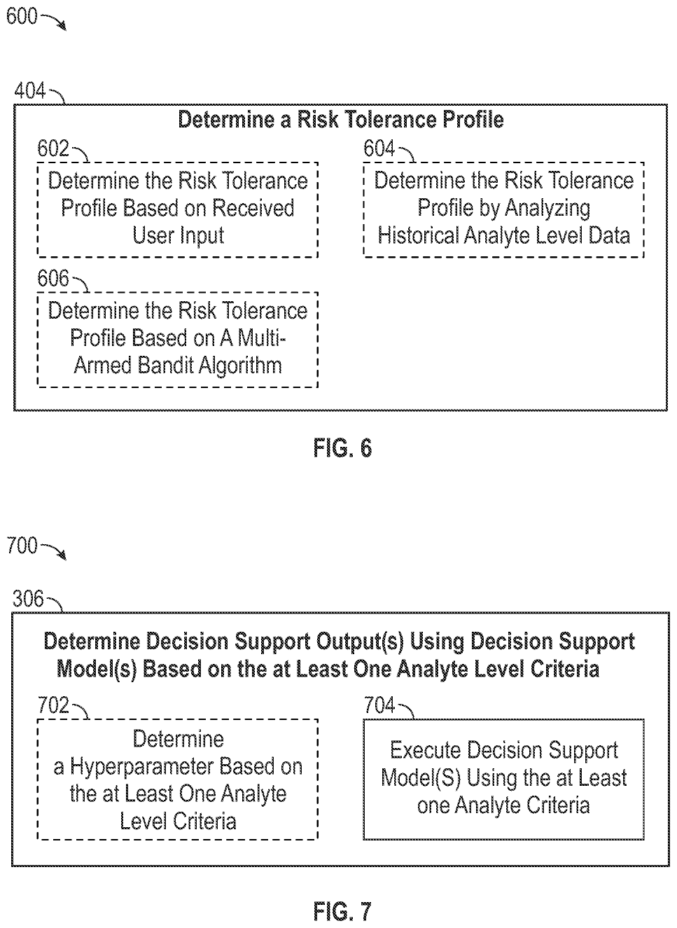

Below, blocks 304 and 306 are described in more detail and by reference to subsequent FIGS. 4-7. As an example, immediately below, block 304 of FIG. 3 is described in more detail in by reference to FIGS. 4-6. In particular, block 304 of FIG. 3 is described in more detail in by reference to blocks 402 and 404 of FIG. 4. Then block 402 of FIG. 4 is described in more detail by reference to blocks 502-508 of FIG. 5 and block 404 of FIG. 4 is described in more detail by reference to blocks 602-606 of FIG. 6. After block 304, block 306 of FIG. 3 is then described in more detail by reference to blocks 702 and 704 of FIG. 7.

### 1. Block 304: Determining at Least One Analyte Level Criteria for a User

As described above in reference to FIG. 3, process 300 may include determining (block 304) at least one analyte level criteria. The analyte level criteria for a user 102 may include at least one of a high-level analyte threshold, a low-level analyte threshold, one or more high-level risk tolerance thresholds, and one or more low-level risk tolerance thresholds. The high-level analyte threshold and the low-level analyte threshold may collectively define an optimal analyte level range for the user 102. The high-level risk tolerance thresholds and the low-level risk tolerance thresholds may collectively define a risk tolerance profile for the user 102. Various example processes for determining the optimal level range and the risk tolerance profile for a user 102 are further described below by reference to FIG. 4. In particular, block 402 of FIG. 4 describes determining an optimal analyte level range for the user while block 404 of FIG. 4 describes determining a risk tolerance profile for the user. An optimal analyte level range and a risk tolerance profile are examples of analyte level criteria.

a. Block 402: Determining the Optimal Analyte Level Range for the User

FIG. 4 is a flow diagram illustrating a process 400 for determining (block 304) at least one analyte level criteria in accordance with certain embodiments of the disclosure. In some embodiments, process 400 for determining (block 304) the at least one analyte level criteria may include determining (block 402) an optimal level range for the user 102 and/or determining (block 404) a risk tolerance profile for the user 102, as further described below. Various techniques can be used to determine (block 402) an optimal analyte level range for a user.

FIG. 5 is a flow diagram illustrating a process 500 for determining (block 402) an optimal analyte level range for a user in accordance with certain embodiments of the disclosure. In some embodiments, the process 500 may include determining (block 502) an optimal analyte level range based on user input (e.g., inputs 127) received from the user 102 and/or determining (block 504) an optimal analyte level range based on healthcare provider input received from a healthcare provider.

For example, the user 102 may provide inputs 127 indicating the high-level analyte threshold and the low-level analyte threshold (i.e., an optimal level range) in a dedicated user interface (UI) of a computing device (e.g., mobile device 107). In some embodiments, to specify the high-level analyte threshold and the low-level analyte threshold, the user 102 may provide a selected textual description of his or her preferred analyte level range (e.g., “tight,” “moderate,” or “wide”) via the application 106 running on the mobile device 107. The textual description may then be mapped to an associated analyte level range (also referred to herein as “analyte level criteria”) by one or more computing devices on the system 100. For example, in embodiments where the DAM 111 runs on the mobile device 107, the textual description may be mapped to an associated analyte level range at the mobile device 107. In some embodiments, the textual description may be stored in the user database 110 and mapped to an associated analyte level range by the user database 110 and/or by the DAM 111 running on one or more servers in network connection with the user database 110. As another example, the user may provide inputs indicating one or more personal health objectives (e.g., keeping the number of hyperglycemia events below a certain number). The computing device may then use historical analyte level trends (e.g., historical glucose concentration trends) of the user to map the objectives to an optimal analyte level range for the user. In some embodiments, historical analyte level trends may be a metric 130 or may be generated using one or more metrics 130. In some embodiments, historical analyte level trends may be generated using one or more inputs 127 and/or a combination of inputs 127 and metrics 130. Moreover, the high-level analyte threshold and the low-level threshold for the user may be determined (block 504) based on data transmitted to the computing device by the user's healthcare provider.

In reference to FIG. 5, the process 500 for determining an optimal level range (block 402) may include determining (block 506) the optimal level range by analyzing historical analyte level data (e.g., metrics 130). In some embodiments, determining (block 506) an optimal analyte level range based on historical analyte level data may be performed based on a user specifically or based on a cohort that includes the user. The determination about whether to use historical analyte level data of the user or the cohort may be based on whether there is enough user-specific historical analyte level data available for the user 102. For example, whether there is enough user-specific historical analyte level available may be based on whether the user-specific historical analyte level covers at least a threshold time period. For example, the threshold time period may be a predetermined time period (e.g., 1 week, 1 month, etc.) or it may be a time period that includes sufficient data (e.g., blood glucose readings) to determine a trend (e.g., glucose trend).

In some embodiments, if historical analyte level data of the user 102 covers at least the threshold time period, the computing device uses trends in the historical analyte level data of the user 102 to determine the high-level analyte threshold and/or the low-level analyte threshold for the user 102. For example, a user's glucose levels may be between 60-160 mg/dl for approximately 90% of any given day. In such an example, the computing device may determine a high-level analyte threshold for the user at the top of the range (e.g., 160 mg/dL) or at some percentage above the top of the range (e.g., 10% above 160 mg/dL). Similarly, the computing device may determine a low-level analyte threshold for the user at the bottom of the range (e.g., 70 mg/dL) or at some percentage below the bottom of the range (e.g., 10% below 70 mg/dL).

If the historical analyte level data for the user 102 does not cover the threshold time period, the computing device uses trends in the historical analyte level data of the cohort to determine the thresholds. For example, a cohort's glucose levels may be between 70-180 mg/dL for approximately 90% of any given day. In such an example, the computing device may determine a high-level analyte threshold for the user at the top of the cohort's range (e.g., 180 mg/dL) or at some percentage above the top of the cohort's range (e.g., 10% above 180 mg/dL). Similarly, the computing device may determine a low-level analyte threshold for the user at the bottom of the cohort's range (e.g., 60 mg/dL) or at some percentage below the bottom of the range (e.g., 10% below 60 mg/dL).

In further reference to FIG. 5, the process 500 for determining an optimal level range (block 402) may include determining (block 508) the optimal level range based various algorithms and/or models such as, but not limited to, a multi-armed bandit algorithm, a contextual multi-armed bandit (CMAB) algorithm, etc. For example, a CMAB model may be utilized to determine an optimal analyte level range for a user 102 or a user cohort based on contextual information as an input. For example, users' contextual information may include demographic information 119, disease progression information 121, medication information 122, inputs 127 (e.g., activity, patient stats, insulin, blood glucose, etc.), metrics 130 (e.g., glucose level, disease stage, etc.), etc.

In some embodiments, the CMAB algorithm may be used to (i) divide a set of users or user cohorts into the exploration subset and an exploitation subset using an exploration ratio, (ii) determine a randomized optimal analyte level range for each user or user cohort in the exploration subset, and (iii) determine the optimal predicted analyte level range for each user or user cohort in the exploitation subset based on a machine learning model using the user's or user cohort's contextual information as input. In some embodiments, the exploration ratio may define a ratio of users or user cohorts that are assigned to the exploration subset. In some embodiments, the exploration ratio may decrease over time. In some embodiments, the exploration ratio may be set to zero after a predefined period of time.

b. Block 404: Determining the Risk Tolerance Profile for the User

As described above, the process 400 for determining (block 304) at least one analyte level criteria may include determining (block 402) an optimal level range for the user 102 (as illustrated in FIG. 5). In addition, the process 400 for determining (block 304) the at least one analyte level criteria may include determining (block 404) a risk tolerance profile for the user 102 (as illustrated in FIG. 6). Various techniques can be used to determine a risk tolerance profile for the user.

FIG. 6 is a flow diagram illustrating a process 600 for determining (block 404) a risk tolerance profile for a user in accordance with certain embodiments of the disclosure. The process 600 may include determining (block 602) the risk tolerance profile for a user 102 based on user input (e.g., inputs 127) received from the user. For example, the user 102 may provide inputs 127 describing the extent of the user's willingness to risk having analyte levels that fall in various hypoglycemic and/or hyperglycemic ranges. To enable the user 102 to provide the inputs 127, a dedicated UI may display various analyte level ranges to the user 102 and ask the user 102 to specify a risk tolerance level for each displayed range. Examples of analyte level ranges that may be displayed to the user 102 include a low hyperglycemia range (e.g., 180-190 mg/dL), a medium hyperglycemia range (e.g., 190-220 mg/dL), and a high hyperglycemia range (e.g., 220 mg/dL or more). Other examples of analyte level ranges that may be displayed to the user 102 include a low hypoglycemia range (e.g., 60-70 mg/dL), a medium hypoglycemia range (e.g., 50-60 mg/dL), and a high hypoglycemia range (e.g., 50 mg/dL less).

After the user 102 provides inputs 127 specifying risk tolerance levels, the computing device (e.g., the mobile device 107) may then use the inputs 127 to determine one or more high-level risk tolerance thresholds and one or more low-level risk tolerance thresholds. For example, the computing device may determine a first high-level risk tolerance threshold for the low hyperglycemia range, a second high-level risk tolerance threshold for the medium hyperglycemia range, and a third high-level risk tolerance threshold for the high hyperglycemia range. Additionally, the computing device may determine a first low-level risk tolerance threshold for the low hypoglycemia range, a second low-level risk tolerance threshold for the medium hypoglycemia range, and a third low-level risk tolerance threshold for the high hypoglycemia range. In this example, the six described risk tolerance thresholds may be determined based on the user-specified risk tolerance levels for the six analyte level ranges displayed to the user 102.

In other embodiments in which the risk tolerance profile is determined (block 602) based on user input, the user 102 may provide inputs 127 describing an acceptable number of high-level analyte events (e.g., hyperglycemia events) and/or low-level analyte events (e.g., hypoglycemia events). For example, the user 102 may specify that he or she wishes to keep the number of daily hyperglycemia events below a certain number. After the user 102 provides inputs 127 describing acceptable number of high-level analyte level events and/or low-level analyte events, the computing device may then determine the risk tolerance profile for the user 102 based on those inputs 127. For example, the computing device may determine one or more high-level risk tolerance thresholds for the user 102 based on the acceptable number of high-level analyte events as provided by the user 102. As another example, the computing device may determine one or more low-level risk tolerance thresholds for the user 102 based on the acceptable number of low-level analyte events as provided by the user 102.

In yet other embodiments in which the risk tolerance profile is determined (block 602) based on user input, the user may provide inputs 127 describing an acceptable time range for high-level analyte events (e.g., hyperglycemia events) and/or a low-level analyte events (e.g., hypoglycemia events). For example, the user 102 may specify that he or she wishes to keep the amount of time he or she experiences hyperglycemia to less than 10 minutes a day. After the user 102 provides inputs 127 describing the acceptable time range for high-level analyte events and/or low-level analyte events, the computing device may then determine (block 602) the risk tolerance profile for the user 102 based on those inputs 127. For example, the computing device may determine (block 602) one or more high-level risk tolerance thresholds for the user 102 based on the acceptable time range for high-level analyte events as provided by the user 102. As another example, the computing device may determine (block 602) one or more low-level risk tolerance thresholds for the user 102 based on the acceptable time range for low-level analyte events as provided by the user 102.

In yet other embodiments in which the risk tolerance profile is determined (block 602) based on user input, sliders (e.g., two sliders) may be displayed to the user 102 via the UI on the computing device (e.g., mobile device 107). For example, a first slider may enable the user 102 to select a high-level risk tolerance threshold and a second slider may enable the user 102 to select a low-level risk tolerance threshold. If the user 102 selects a value for the high-level risk tolerance threshold using the first slider, the second slider may be updated to change the selected value for the low-level risk tolerance threshold. The new selected value of the second slider may be computed using a trade-off model that may be configured to predict how changes in one risk threshold affect the other risk threshold. For example, in some embodiments, a trade-off model may be based on historical individual glucose data (if available), cohort glucose data (if available), or population-level glucose data. The trade-off model/analysis may determine to what degree less time above range is associated with more time below range, or reversely, to what degree more time above range is associated with less time below range. In various embodiments, after the user 102 selects the value for the high-level risk tolerance threshold using the first slider, the trade-off model may predict how the selection affects the value of the low-level risk tolerance threshold and thus may determine the new selected value of the second slider. Similarly, if the user 102 selects a value for the low-level risk tolerance threshold using the second slider, the first slider may be updated to change the selected value for the high-level risk tolerance threshold to a value computed by the trade-off model.

In further reference to FIG. 6, the process 600 may also include determining (block 604) the risk tolerance by analyzing historical analyte level data. For example, certain other techniques involve determining (block 604) the risk tolerance profile for a user based on trends in the historical analyte level data (e.g., metrics 130) for the user 102 and/or for a cohort including the user 102. For example, the historical analyte level data for the user 102 may indicate that the user 102 sometimes intervenes (e.g., with insulin) if his or her glucose levels are between 190-200 mg/dL, but always intervenes if he or her glucose levels are between 201-220 mg/dL. Based on these indications in the historical analyte level data for the user 102, the computing device may determine a higher high-level risk threshold for the range 201-220 mg/dL than for the range 190-200 mg/dL.

In further reference to FIG. 6, the process 600 may also include determining (block 606) the risk tolerance based on various algorithms or models such as, but not limited to, a multi-armed bandit algorithm, a contextual multi-armed bandit (CMAB) algorithm, etc. For example, a CMAB algorithm may be utilized to determine a risk tolerance profile for a user or a user cohort based on contextual information as an input. For example, users' contextual information may include demographic information 119, disease progression information 121, medication information 122, inputs 127 (e.g., activity, patient stats, insulin, blood glucose, etc.), metrics 130 (e.g., glucose level, disease stage, etc.), etc.

In some embodiments, the CMAB algorithm may be used to (i) divide a set of users or user cohorts into the exploration subset and an exploitation subset using an exploration ratio, (ii) determine a randomized risk tolerance profile for each user or user cohort in the exploration subset, and (iii) determine the risk tolerance profile for each user or user cohort in the exploitation subset based on based on the user's or user cohort's contextual information. In some embodiments, the exploration ratio may define a ratio of users or user cohorts that are assigned to the exploration subset. In some embodiments, the exploration ratio may decrease over time. In some embodiments, the exploration ratio may be set to zero after a predefined period of time.

As an example of assigning a randomized risk tolerance profile for each user or user cohort in the exploration subset, a first user or user cohort can be assigned a risk tolerance profile to keep the amount of time hyperglycemia is experienced to less than 10 minutes a day and the second user or cohort may be assigned a risk profile to keep the amount of time hyperglycemia is experienced to less than 5 minutes a day.

### 2. Block 306: Determining Decision Support Outputs Using Decision Support Models Based on the at Least One Analyte Level Criteria

As described above in reference to FIG. 3, process 300 may include determining (block 306) one or more decision support outputs using one or more decision support models based on the at least one analyte level criteria. For example, the at least one analyte level criteria (e.g., a set of analyte level criteria for a user) determined in block 304, may be used in combination with one or more decision support models to determine one or more decision support outputs for the user.

FIG. 7 is a flow diagram illustrating a process 700 for determining (block 306) at least one decision support output using decision support models based on the at least one analyte level criteria in accordance with certain embodiments of the disclosure. Various techniques may be used for using an analyte level criteria for a user in conjunction with a decision support model to determine (block 306) a decision support output for the user. For example, in some embodiments, the process 700 may include determining (block 702) one or more hyperparameters of a decision support model based on the analyte level criteria. In some embodiments, a hyperparameter of a decision support model may be a value that defines the operational logic of the decision support model. In some embodiments, a hyperparameter may be considered a part of the model inputs along with data to train, validate, and test on the model. In some embodiments, a hyperparameter may be considered a part of a decision support model definition itself, where one or more hyperparameters may be chosen (or optimized) to define a decision support model. For example, a hyperparameter may be a hyperglycemia threshold hyperparameter for a hyperglycemia warning model. As another example, the analyte level criteria for a user may be provided as model inputs to a decision support model to determine the decision support output for the user.

In reference to FIG. 7, the process 700 may include executing (block 704) one or more decision support models using the at least one analyte criteria. In some embodiments, executing (block 704) a decision support model may include determining a decision support output for a user based on input data that includes the high-level analyte threshold, the low-level analyte threshold, the high-level risk tolerance thresholds, and/or the low-level risk tolerance thresholds for the user 102. In some embodiments, executing (block 704) a decision support model may include determining a decision support output for a user by using: (i) the high-level analyte threshold and the low-level analyte threshold (e.g., optimal level range) for the user 102 as input data, and/or (ii) the high-level risk tolerance thresholds and the low-level risk tolerance thresholds (e.g., risk tolerance profile) for the user 102 as hyperparameters for the decision support model.

In some embodiments, a decision support model may comprise a scoring sub-model and a decision sub-model. The scoring sub-model may be configured to process input data including the at least one analyte level criteria for a user 102 to determine a set of risk scores for the user 102. Each determined risk score may describe a predicted likelihood that the user 102 experiences a particular condition (e.g., a high-level analyte condition such as hyperglycemia or a low-level analyte condition such as hypoglycemia) in a future time period (e.g., in the next eight hours). The scoring sub-model may be a trained machine learning model characterized by one or more trained parameters. In some embodiments, a trained parameter may be a value that affects the operational logic of a decision support model and may be determined by training the decision support model.

The decision sub-model may be configured to select a decision support output for the user 102 from a set of potential decision support outputs based on the determined risk scores for the user 102. In some embodiments, the decision sub-model may be characterized by one or more hyperparameters that may define risk tolerance thresholds for the one or more risk scores. Each potential decision support output may be associated with a respective subset of the risk tolerance thresholds that may be defined by the hyper-parameters of the decision sub-model. If the risk scores of a user 102 satisfy the risk tolerance thresholds that are associated with a potential decision support output, then the decision sub-model may select the potential decision support output and provide the decision support output to the user 102. In certain embodiments, the hyperparameters of the decision sub-model may be determined based on at least one analyte criteria for the user.

For example, the scoring sub-model of a hyperglycemia warning model may be configured to determine a hyperglycemia risk score for a user 102, while the decision sub-model of the hyperglycemia warning model may be configured to determine that a hyperglycemia warning should be presented to the user 102 if the hyperglycemia risk score for the user 102 satisfies (e.g., falls above) a hyperglycemia risk tolerance threshold. In this example, the input data to the scoring sub-model may include glucose concentration measurements of the user 102 and a hyperglycemia threshold for the user 102. Moreover, the hyperglycemia risk tolerance threshold may be the sole hyperparameter of the decision sub-model, while the two potential decision support outputs of the hyperglycemia warning model include a “warning” output that is configured to cause a hyperglycemia warning to be presented to a user and a “no warning” output that is configured to prevent the hyperglycemia warning from being presented to the user. As this example illustrates, when a decision support model determines a decision support output for a user, both the input data (in this case, the hyperglycemia threshold) for the scoring sub-model and the hyperparameters (in this case, the hyperglycemia risk tolerance threshold) for the decision sub-model may be defined by the at least one analyte level criteria for the user, which are the hyperglycemia threshold and the hyperglycemia risk tolerance threshold.

As another example, the scoring sub-model of a sleep advisor model may be configured to determine a hyperglycemia risk score and a hypoglycemia risk score for a user 102. The decision sub-model of the sleep advisor model may be configured to select a sleep pattern from a set of potential sleep patterns for recommending to the user 102. A sleep pattern may be selected when the risk scores of the user 102, as determined by the scoring sub-model, satisfy both a hyperglycemia risk tolerance threshold and a hypoglycemia risk tolerance threshold for the selected sleep pattern. The decision sub-model may be characterized by a set of hyperparameters that define two risk tolerance thresholds for each one of the potential sleep patterns: a high-level risk tolerance threshold and a low-level risk tolerance threshold.

For example, the set of potential sleep patterns may include an 8-hour sleep pattern and a 10-hour sleep pattern. In this example, the 8-hour sleep pattern may be recommended to a user 102 if the hyperglycemia risk score for the user satisfies a first high-level risk tolerance threshold and the hypoglycemia risk score for the user 102 satisfies a first low-level risk tolerance threshold, while the 10-hour sleep pattern may be recommended to the user 102 if the hyperglycemia risk score for the user satisfies a second high-level risk tolerance threshold and the hypoglycemia risk score for the user 102 satisfies a second low-level risk tolerance threshold. The risk tolerance thresholds for potential sleep patterns may be determined based on the risk tolerance profile for the user 102 as described by the user's analyte level criteria. As this example illustrates, when a decision support model determines a decision support output for a user 102, both the input data (in this case, the hyperglycemia threshold and the hypoglycemia threshold) for the scoring sub-model and the hyperparameters (in this case, the hyperglycemia risk tolerance thresholds and the hypoglycemia risk tolerance thresholds for potential sleep patterns) for the decision sub-model may be defined by analyte level criteria for the user.

The implementations and examples described herein are merely exemplary and various implementations as appropriate to the requirements of a specific application may be utilized in accordance with certain embodiments described herein. For example, another variation is a model that outputs a pre-bedtime carb consumption (or exercise/insulin) recommendation, if risk of overnight hypoglycemia (or hyperglycemia) is predicted. These recommendations could have additional hyperparameters (on top of risk threshold) related to the type/amount of recommended exercise/carbs/insulin.

In some embodiments, the decision support models may include a gradual transition model (also referred to as a “nudging” model). In various embodiments, a gradual transition model may be configured to determine a sequence of analyte levels to recommend to the user 102 over a sequence of future time periods to help and encourage the user to gradually transition his or her analyte values from a current analyte value range to an optimal analyte value range. In some embodiments, the gradual transition model is further configured to recommend a sequence of actions for the user 102 to perform over the sequence of future time periods to help and encourage the user 102 to gradually transition his or her analyte values from the current analyte value range to the optimal analyte value range. In some embodiments, the gradual transition model may recommend different optimal analyte value ranges for different periods of a day. In some embodiments, input data to the gradual transition model may include data describing the current analyte value range for the user 102 and data describing the optimal analyte value range for the user 102. The current analyte value range for the user 102 may be determined by monitoring analyte concentration measurements for the user 102 over a period or may be set to a default range. The optimal analyte value range may be determined based on the high-level analyte threshold and the low-level analyte threshold for the user 102 as indicated by the analyte level criteria for the user 102.

The gradual transition model may be configured to iteratively guide the user 102 from the current analyte level range for the user 102 to the optimal analyte level range for the user 102. For example, the model may recommend a glucose range of 70-220 mg/dL in a first month, 70-200 mg/dL in a second month, 70-180 mg/dL in a third month, and 70-160 mg/dL in a fourth month. In some embodiments, to determine each recommended analyte level, the gradual transition model excludes those analyte levels whose risk scores fail to satisfy the risk tolerance profile of the user 102, as indicated by the analyte level criteria for the user 102. For example, the gradual transition model may select a recommended analyte level for a user 102 from a set of analyte levels whose hyperglycemia risk scores satisfy the hyperglycemia risk tolerance threshold for the user 102 and whose hypoglycemia risk scores satisfy the hypoglycemia risk tolerance threshold for the user 102.

In some embodiments, the gradual transition model may determine the magnitudes and timings of recommended changes in analyte levels. To determine the magnitude and timing of a recommended analyte level change, the gradual transition model may use the directions and magnitudes of the user's recent changes in analyte levels to determine how aggressively to change the recommended analyte levels of the user 102. For example, recent large improvements in glucose concentration measurements may be used to infer a momentum in the user 102's behavior and determine a more aggressive recommended change in analyte levels of the user 102. In some embodiments, the magnitude and timing of recommended changes in analyte levels of a user 102 may be determined using contextual data (e.g., demographic data, such as age data) associated with the user 102 and by utilizing a CMAB algorithm, such as a multi-objective CMAB algorithm.

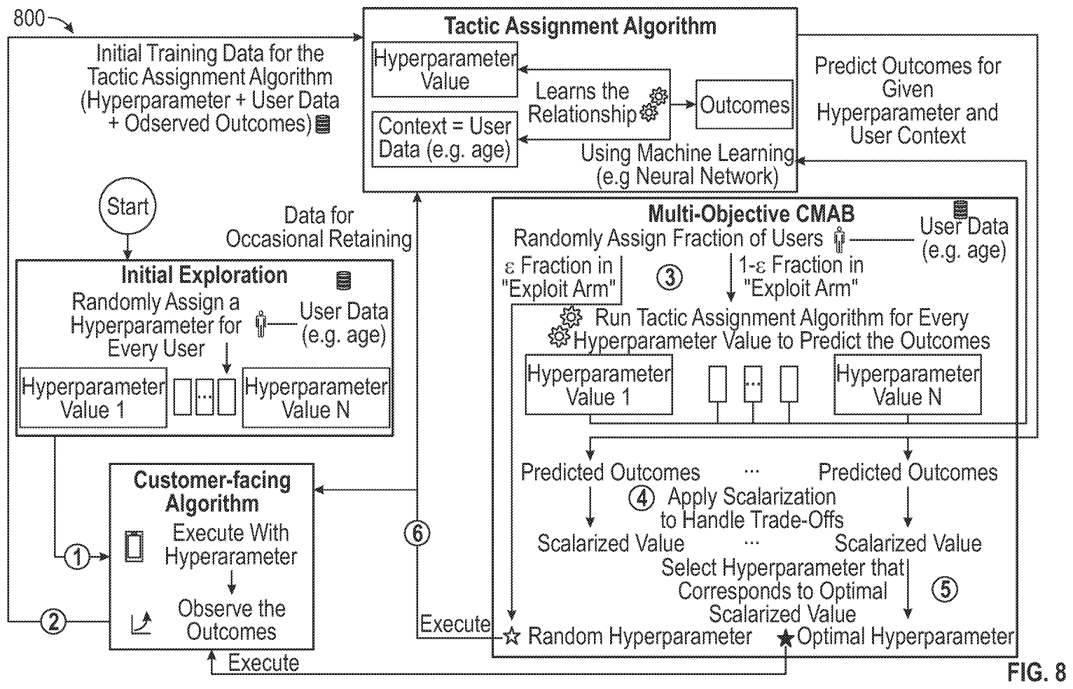

FIG. 8 is a flow diagram illustrating an exemplary operational process 800 of a CMAB algorithm in accordance with certain embodiments of the disclosure. The process 800 may include assigning (Step 1) a random set of hyperparameters to each user. For example, during an initial exploration, the CMAB algorithm may assign each user a hyperglycemia threshold (e.g., hyperglycemia threshold value 1=180 mg/dL, hyperglycemia threshold value 2=185 mg/dl, hyperglycemia threshold value 3=190 mg/dL, hyperglycemia threshold value 4=195 mg/dl, etc.). The process 800 may also include determining (Step 2) decision support outputs based on the assigned set of hyperparameters, observing outcomes associated with the decision support outputs, and training an outcome prediction model based on the observed data.

For example, for each user, a customer-facing algorithm may recommend a sequence of analyte levels over a sequence of future time periods based on the assigned hyperglycemia threshold. For example, for a user that is assigned hyperglycemia threshold 1, the sequence of analyte levels over a sequence of future time periods may be aggressive (e.g., glucose range 70-220 mg/dL in week 1, 70-200 mg/dL in week 2, 70-180 mg/dL in week 3, and 70-160 mg/dL in week 4). However, for a user that is assigned hyperglycemia threshold 4, the sequence of analyte levels over a sequence of future time periods may be less aggressive (e.g., glucose range 70-220 mg/dL in month 1, 70-200 mg/dL in month 2, 70-180 mg/dL in month 3, and 70-160 mg/dL in week 4).

In another example, for each user, a customer-facing algorithm may recommend a sequence of actions to perform over a sequence of future time periods. For example, for a user that is assigned hyperglycemia threshold 1, the sequence of actions may be aggressive (e.g., take 5000 steps per day in week 1, take 6000 steps per day in week 2, take 7000 steps per day in week 3, and take 8000 steps per day in week 4). However, for a user assigned hyperglycemia threshold 4, the sequence of actions may be less aggressive (e.g., take 5000 steps per day in week 1, take 5500 steps per day in week 2, take 6000 steps per day in week 3, and take 6500 steps per day in week 4)). The CMAB algorithm may observe the outcomes associated with the decision support outputs and train the outcome prediction model based on the observed data.

Further, the process 800 may include dividing (Step 3) the users into an exploration subset and an exploitation subset and determining (Step 4) scaled outcomes for users in the exploitation subset by scaling predicted outcomes determined using the outcome prediction model. For example, the CMAB algorithm may place users in an exploration subgroup that will continue to be assigned a random hyperparameter (e.g., one of hyperglycemia thresholds 1-4). Further, the CMAB algorithm may place users in an exploitation subgroup and users in the exploitation subgroup may be assigned a hyperparameter based on each user's contextual information and the model's learned relationship between context (e.g., user's age) and a certain hyperparameter value (e.g., one of hyperglycemia thresholds 1-4), with the outcome. The learned relationship allows the CMAB to predict outcomes such as, but not limited to, the success rate of a recommendation of a particular sequence of analyte levels over a sequence of future time periods or a recommendation of a particular sequence of actions over a sequence of future time periods for a particular user. In certain embodiments, the predicted outcomes may be represented by scalarized values and the CMAB may select hyperparameters that correspond to optimal scalarized values for users in the exploitation subgroup. In other words, the process 800 may include determining (Step 5) optimal hyperparameter sets for the users in exploitation subset based on the scaled outcomes and assigning the optimal hyperparameters sets to those users. Furthermore, the process 800 may include randomly assigning (Step 6) hyperparameters to users in the exploration subset and using this data and observed outcomes to retrain an outcome prediction model. In certain embodiments, the process 800 may repeat Steps 2-6.

### Example Apparatus for Determining Decision Support Outputs Using User-Specific Analyte Level Criteria

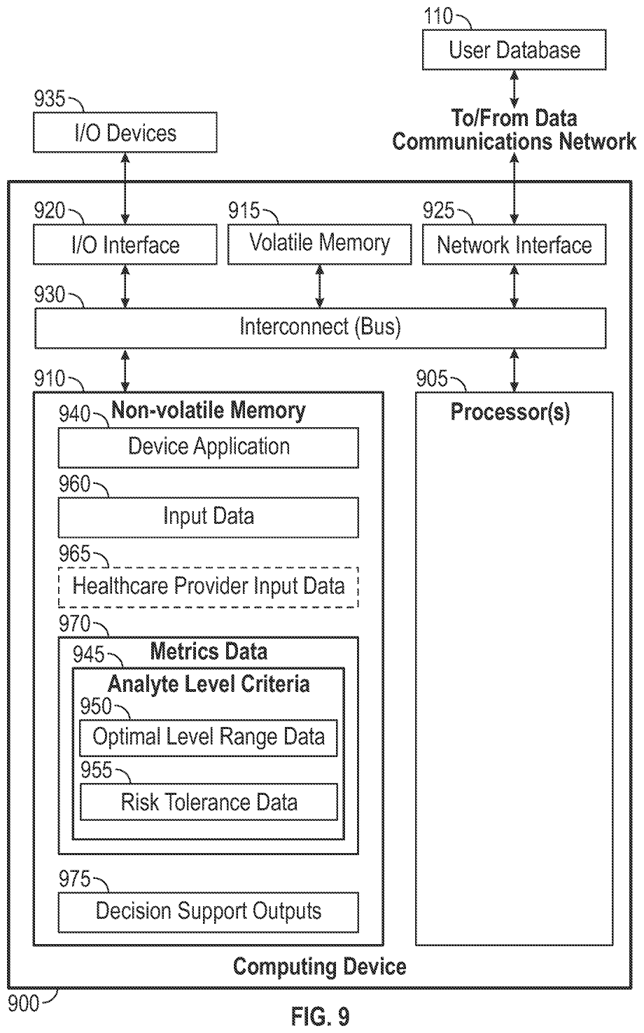

FIG. 9 is a block diagram depicting an example computing device 900 configured to determine decision support outputs using user-specific analyte level criteria, according to certain embodiments disclosed herein. Although depicted as a single physical device, in embodiments, computing device 900 may be implemented using virtual device(s), and/or across a number of devices, such as in a cloud environment. As illustrated, computing device 900 includes a one or more processor(s) 905, non-volatile memory 910, volatile memory 915, a network interface 925, and one or more input/output (I/O) interfaces 920. In the illustrated embodiment, processor 905 retrieves and executes programming instructions stored in the non-volatile memory 910 and/or the volatile memory 915, as well as stores and retrieves data residing in the non-volatile memory 910 and/or the volatile memory 915. In certain embodiments, non-volatile memory 910 is configured to store instructions (e.g., computer-executable code, device application 940) that when executed by processor(s) 905, cause processor(s) 905 to perform the processes and/or operations described herein and illustrated in FIGS. 3-8. In certain embodiments, non-volatile memory 910 stores code for executing the functions of the DAM 111, decision support engine 112, and/or the application 106. Note that computing device 900 may be configured to perform the functions of only one of the DAM 111, decision support engine 112, and/or the application 106, in which case additional system(s) may be used for performing the functions of the others.

Processor(s) 905 is generally representative of a single central processing unit (CPU) and/or graphics processing unit (GPU), multiple CPUs and/or GPUs, a single CPU and/or GPU having multiple processing cores, and the like. Volatile memory 915 is generally included to be representative of a random access memory (RAM). Non-volatile memory 910 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 935 (such as keyboards, monitors, etc.) can be connected via the I/O interface(s) 920. Further, via network interface 925, computing device 900 can be communicatively coupled with one or more other devices and components, such as user database 110. In certain embodiments, computing device 900 is communicatively coupled with other devices via a network, which may include the Internet, local network(s), and the like. The network may include wired connections, wireless connections, or a combination of wired and wireless connections. As illustrated, processor(s) 905, non-volatile memory 910, volatile memory 915, network interface 925, and I/O interface(s) 920 are communicatively coupled by one or more bus interconnects 930. In certain embodiments, computing device 900 is a server executing in an on-premises data center or a cloud environment. In certain embodiments, the computing device 900 is a user's mobile device.

In the illustrated embodiment, the non-volatile memory 910 may include a device application 940 that configures the processor(s) 905 to perform various processes and/or operations in determining decision support outputs 975 using user-specific analyte criteria, as described above. In some embodiments, the device application 940 may perform the functions of the DAM 111, the decision support engine 112, and/or the application 106. As described above in reference to FIGS. 3-8, the computing device 900 may be configured to determine and/or store at least one analyte level criteria 945. In some embodiments, the at least one analyte level criteria 945 may include optimal level range data 950 and/or risk tolerance profile data 955, as further described above. In addition, the computing device 900 may be configured to receive and/or store input data 960 (e.g., inputs 127) and/or healthcare provider input data 965. Furthermore, the computing device 900 may be configured to receive and/or generate metrics data 970 (e.g., metrics 130), as described above. In certain embodiments, metrics data 970 may include the at least one analyte level criteria 945 which includes the optimal level range data 950 and/or the risk tolerance data 955.

Each of these non-limiting examples can stand on its own or can be combined in various permutations or combinations with one or more of the other examples. The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel.” “perpendicular,” “round,” or “square,” are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A non-transitory computer readable storage medium storing a program comprising instructions that, when executed by at least one processor of a computing device, cause the at least one processor to perform operations including:
receiving sensor data generated by an analyte sensor configured to monitor at least one analyte;
determining at least one analyte level criteria for a user for the at least one analyte;
determining, using a decision support model, at least one decision support output based on the at least one analyte level criteria; and
providing the at least one decision support output to the user.

2. The non-transitory computer readable storage medium of claim 1, wherein the at least one analyte level criteria is an optimal level range for the at least one analyte.

3. The non-transitory computer readable storage medium of claim 2, wherein the optimal level range comprises a high-level analyte threshold that defines an upper boundary for the at least one analyte and a low-level analyte threshold that defines a lower boundary for the at least one analyte.

4. The non-transitory computer readable storage medium of claim 3, wherein the operations further comprise:
receiving user input that indicates the high-level analyte threshold and the low-level analyte threshold, wherein the high-level analyte threshold and the low-level analyte threshold are determined based on the user input.

5. The non-transitory computer readable storage medium of claim 3, wherein the optimal level range is determined by:
defining a threshold time period; and
upon determining that the sensor data covers the threshold time period, generating trends using the sensor data to determine the high-level analyte threshold and the low-level analyte threshold.

6. The non-transitory computer readable storage medium of claim 3, wherein the optimal level range is determined by:
defining a threshold time period; and
upon determining that the sensor data does not cover the threshold time period, generating trends using sensor data of a cohort to determine the high-level analyte threshold and the low-level analyte threshold.

7. The non-transitory computer readable storage medium of claim 1, wherein the at least one analyte level criteria is a risk tolerance profile for the user.

8. The non-transitory computer readable storage medium of claim 7, wherein the risk tolerance profile comprises a high-level risk tolerance threshold defining a user's willingness to risk having analyte levels exceed a recommended high analyte level range, and a low-level risk tolerance threshold defining the user's willingness to risk having the analyte levels fall below a recommended low analyte level range.

9. A method for determining decision support outputs using user-specific analyte level criteria, the method comprising:
receiving sensor data generated by an analyte sensor configured to monitor at least one analyte;
determining at least one analyte level criteria for a user for the at least one analyte;
determining, using a decision support model, at least one decision support output based on the at least one analyte level criteria; and
providing the at least one decision support output to the user.

10. The method of claim 9, wherein the at least one analyte level criteria is an optimal level range for the at least one analyte.

11. The method of claim 10, wherein the optimal level range comprises a high-level analyte threshold that defines an upper boundary for the at least one analyte and a low-level analyte threshold that defines a lower boundary for the at least one analyte.

12. The method of claim 11, further comprising:
receiving user input that indicates the high-level analyte threshold and the low-level analyte threshold, wherein the high-level analyte threshold and the low-level analyte threshold are determined based on the user input.

13. The method of claim 9, wherein the at least one analyte level criteria is a risk tolerance profile for the user.

14. The method of claim 13, wherein the risk tolerance profile comprises a high-level risk tolerance threshold defining a user's willingness to risk having analyte levels exceed a recommended high analyte level range, and a low-level risk tolerance threshold defining the user's willingness to risk having the analyte levels fall below a recommended low analyte level range.

15. A computing device for determining decision support outputs using user-specific analyte level criteria, the computing device comprising:
a network interface;
a processor operatively connected to the network interface;
a memory storing a program comprising instructions that, when executed by the processor, cause the computing device to:
receive, using the network interface, sensor data generated by an analyte sensor configured to monitor at least one analyte;
determine at least one analyte level criteria for a user for the at least one analyte;
determine, using a decision support model, at least one decision support output based on the at least one analyte level criteria; and
provide the at least one decision support output to the user.

16. The computing device of claim 15, wherein the at least one analyte level criteria is an optimal level range for the at least one analyte.

17. The computing device of claim 16, wherein the optimal level range comprises a high-level analyte threshold that defines an upper boundary for the at least one analyte and a low-level analyte threshold that defines a lower boundary for the at least one analyte.

18. The computing device of claim 17, wherein the computing device is further configured to:
receive user input that indicates the high-level analyte threshold and the low-level analyte threshold, wherein the high-level analyte threshold and the low-level analyte threshold are determined based on the user input.

19. The computing device of claim 15, wherein the at least one analyte level criteria is a risk tolerance profile for the user.

20. The computing device of claim 19, wherein the risk tolerance profile comprises a high-level risk tolerance threshold defining a user's willingness to risk having analyte levels exceed a recommended high analyte level range, and a low-level risk tolerance threshold defining the user's willingness to risk having the analyte levels fall below a recommended low analyte level range.
